(19) United States
(12) Patent Application Publication
(10) Pub. No.: US 2026/0245695 A1
(43) Pub. Date: Aug. 20, 2026

(54) SYSTEMS AND METHODS FOR AUTOMATICALLY REVIEWING PRESCRIPTION REQUESTS

(71) Applicant: Express Scripts Strategic Development, Inc., St. Louis, MO (US)

(72) Inventors: Daniel C. Casper, Ellisville, MO (US); Robert Oakley, West Hartford, CT (US); Urvashi Patel, Windermere, FL (US); Christopher R. Markson, Hawthorne, NJ (US); Pritesh J. Shah, Paramus, NJ (US); Abbai Manyam, Montville, NJ (US)

(21) Appl. No.: 19/543,743

(22) Filed: Feb. 18, 2026

Related U.S. Application Data
(60) Provisional application No. 63/759,811, filed on Feb. 18, 2025.

(51) Int. Cl. G16H 20/13; G16H 10/60; G16H 40/60

(52) U.S. Cl. CPC G16H 20/13; G16H 10/60; G16H 40/60

(57) ABSTRACT

A method and system for automatically reviewing a prescription request using a large language model (LLM) system is provided. The method can include receiving, by the LLM system, a request to locate medical data associated with a medical history of a patient from one or more data sources. The medical history can include patient data, historical prescription data, and medical notes provided by a medical professional. The LLM system can provide the medical data that is located by the LLM system to one or more processors. A summary of the medical history of the patient can be generated by the one or more processors, and a recommendation for approving or denying the prescription request can be determined based at least in part on the summary of the medical history of the patient.

### CROSS-REFERENCE TO RELATED APPLICATIONS

This application claims priority to U.S. Provisional Application No. 63/759,811 (filed 18 Feb. 2025), the entire disclosure of which is incorporated herein by reference.

### TECHNICAL FIELD

The inventive subject matter generally relates to the technical field of automated systems, and more specifically to subject matter involving the use of large language models (LLMs) to analyze medical data and make decisions regarding prescription request approvals.

### BACKGROUND

Current methods for reviewing pharmaceutical prescription requests can require a call center agent to review medical history of the patient prior to issuing approval or denial of the request. The amount of medical data that is to be reviewed by the agent can be substantial, inconsistent (e.g., data provided by different providers, by different pharmacies, etc.) and require a substantial amount of time for the manual review process to be completed. The human agents at these call centers can be required to handle a wide variety of inquiries from members (e.g., of benefit plans), providers (e.g., of services, such as healthcare providers), customers, or the like, which call into the centers. These agents can spend a significant amount of time scavenging for data across many sources to serve the callers. Agents may be required to manually gather data from various tools and applications to understand the context of the data, fetch the data, aggregate the data, and then communicate the data back to the caller. This manual process places a significant burden on the agents, requiring extensive training and leading to inefficiencies in handling customer inquiries.

In some instances, a benefit plan or legislative/regulatory requirement may specify prerequisites that are to be met prior to the prescription being covered by a benefits plan. In other instances, some prescription medications may only be approved to be covered based on a medical diagnosis by a physician. As another example, some prescription medications may only be approved to be covered based on biometric data or test results of the patient (e.g., within a predetermined window of time).

A need exists for improving the workflow of call center agents to respond to caller inquiries for a prescription medication request and improving the accuracy of the approval process.

### BRIEF SUMMARY

In one example, a method for automatically reviewing a prescription request using an LLM system is provided. The method can include receiving, by the LLM system, a request to locate medical data associated with a medical history of a patient from one or more data sources. The medical history can include patient data, historical prescription data, and medical notes provided by a medical professional. The LLM system can provide the medical data that is located by the LLM system to one or more processors. A summary of the medical history of the patient can be generated by the one or more processors, and a recommendation for approving or denying the prescription request can be determined based at least in part on the summary of the medical history of the patient.

In another example, a system for reviewing a prescription request is provided. The system can include an LLM system that can receive a request to locate medical data associated with a medical history of a patient from one or more data sources. The medical history can include patient data, historical prescription data, and medical notes provided by a medical professional. The system can further include one or more processors that can receive the medical data that is located by the LLM system. The processors can generate a summary of the medical history of the patient and determine a recommendation for approving or denying the prescription request based at least in part on the summary of the medical history of the patient.

In another example, method for automatically reviewing a prescription request using an LLM system is provided. The method can include receiving, by the LLM system, a request to locate medical data associated with a medical history of a patient from one or more data sources. The medical history can include patient data, historical prescription data, and medical notes provided by a medical professional. The method can further include providing, by the LLM system, the medical data that is located by the LLM system to one or more processors. The one or more processors can generate a summary of the medical history of the patient and determine a recommendation for denying the prescription request based at least in part on the summary of the medical history of the patient. A display device can display the summary of the medical history and the recommendation to an operator to deny the prescription request. The recommendation that is determined by the processors can be overridden and approved by the operator, and the prescription request can be fulfilled.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some embodiments are illustrated by way of example, and not limitation, in the figures of the accompanying drawings in which:

FIG. 1 illustrates one example of a call center operations system;

FIG. 2 illustrates one example of a machine learning (ML)/artificial intelligence (AI) system;

FIG. 3 illustrates a flowchart of one example for reviewing a prescription request at a call center; and

FIG. 4 illustrates one example of a pharmacy fulfillment device that can be used to process and fulfill prescriptions and prescription orders.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

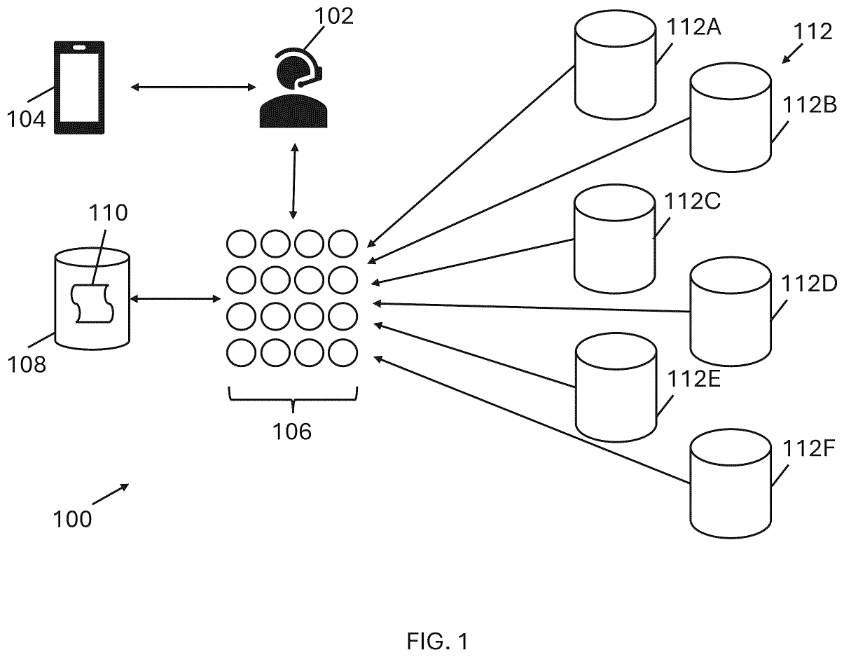

FIG. 1 illustrates one example of a call center operations system 100. The operations system 100 includes one or more (or many) call center agent devices 102 that receive inquiries from customer or member devices 104. Each of the devices 102, 104 can represent hardware circuitry that includes and/or is connected with one or more processors for performing the operations described in connection with each of the devices 102, 104. The processor(s) can include one or more integrated circuits, application-specific integrated circuits (ASICs), microprocessors, field programmable gate arrays, etc. The devices 102, 104 include communication circuitry such as antennas, modems, converters, etc. that can communicate with each other via or over one or more computerized communication networks (e.g., the Internet, one or more intranets, wide area networks, local area networks, etc.).

The operations center 100 includes an artificial intelligence (AI) system, a machine learning (ML) system, and/or an artificial neural network (ANN) 106 that performs operations described herein. The ANN 106 can assist a call center agent via the agent device 102. For example, callers may submit inquiries to the call center using the member devices 104. These inquiries can be phone calls, text messages or other messaging services, online chats, or the like, between the devices 102, 104. The inquiries can include a variety of questions seeking assistance, such as seeking information regarding one or more benefits provided by a pharmacy benefit plan. For example, members may contact the agent device 102 asking whether a prescription for medication is covered by benefits under a benefit plan, to request to fill or refill a prescription for medication, what remaining information is required for preauthorization for the fulfillment of a prescription medication, how to appeal the denial of a prescription request, and so on.

The AI system, ML system, or ANN 106 of the operations system 100 can receive inputs provided to and/or by the agent device 102. These inputs can include the ANN 106 recording an audio call between the devices 102, 104 and transcribing the call into text or alphanumeric text. The inputs can include the typed messages sent from the member device 104 or between the devices 102, 104. The inputs can include keystrokes, electronic mouse inputs, electronic stylus inputs, detected touches on a touchscreen, or the like, on the agent device 102. The ANN 106 can record these and/or other inputs in a tangible and non-transitory computer readable storage medium 108, or computer memory 108. This memory 108 can represent one or more computer hard drives, servers, databases, optical discs, or the like.

The ANN 106 can provide the recorded inputs to a large language model (LLM) system 110 of the operations system 100. The LLM system 110 may be a model stored in the memory 108 or in another tangible and non-transitory computer readable storage medium. The LLM system 110 can be trained to identify the context of the inquiry from the member device 104, as well as information that may be responsive to the inquiry. The LLM system 110 can receive the recorded inputs of the transcribed call, messages, keystrokes by the agent on the agent device 102, etc. and, based on these inputs, determine what information will assist the agent in rectifying or otherwise responding to the inquiry. For example, the LLM system 110 can examine the recorded inputs and decide that the inquiry relates to a prescription medication request. These inputs may include terms such as “prescription,” “refill,” “fill,” names of medications, names of providers, or the like.

Once the purpose of the inquiry is identified by the LLM system 110, the LLM system 110 can output the inquiry purpose to the ANN 106. The ANN 106 can then automatically access one or more, or several, different databases 112 (e.g., databases 112A-G) to obtain information helpful to responding to the inquiry. For example, the caller may be communicating with the call center agent inquiring about filling and/or refilling a prescription medication, and the information obtained by the ANN 106 may be helpful in assisting the agent at the call center in reviewing the prescription request and determining if the request should be approved, should be denied, or if more information is needed in making the decision. Information associated with a medical history of a patient may be critical in determining whether the prescription medication should be filled or not. The medical history of the patient may include, but is not limited to, patient data, historical prescription data, historical purchase data associated with OTC medications or the like, office visits, medical notes, or the like.

The different databases may be accessed by the ANN 106 to locate and/or retrieve medical data associated with the prescription medication request by the caller. While seven databases 112 are shown, fewer or more databases 112 may be accessible to the ANN 106. These databases 112 can each represent a tangible and non-transitory computer readable storage medium, or can represent different sections or partitions within the same storage medium. The databases 112 may be maintained by the operations system 100 or by a third party, such as a healthcare provider, hospital system, or the like. The databases may store information about the caller such as a patient's medical history, a patient's historical prescription medication data, medical notes, office visits, or the like.

The database 112A can represent a benefit claims database that stores medical claims data. The claims data can identify the members with whom the claims are associated, and can include information regarding pharmacy claims adjudicated by the pharmacy benefit manager under a drug benefit program provided by the pharmacy benefit manager for one, or more than one, plan sponsors. The claims data can identify the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copay/coinsurance amount, rebate information, and/or member eligibility, and the like. Additional information may be included.

The database 112B can represent a provider database storing healthcare provider data. This data can indicate the names, locations, practice areas, etc. of different healthcare providers, whether the providers are within a provider network of a benefit plan for members or outside of the network, and the like. Optionally, this data can include details of medications previously prescribed by providers to the patient (e.g., names of medications, dosages, timeline of administering medications, etc.), medical notes associated with office visits by the patient to different providers (e.g., dates, times, and locations of the visit), biometric data and/or medical test results from the office visits, or the like. Optionally, this data can indicate prior claims, procedures, preauthorizations, failed preauthorizations, or the like, involving different healthcare providers.

The database 112C can represent a member database storing member data. The member data can include information regarding the members associated with the pharmacy benefit manager. The information stored as member data may include personal information, personal health information, protected health information, and the like. Examples of the member data include demographic information such as names, addresses, telephone numbers, e-mail addresses, prescription drug histories, etc., and the like. The member data may include a plan sponsor identifier that identifies the plan sponsor associated with the member and/or a member identifier that identifies the member to the plan sponsor. The member data may include a member identifier that identifies the plan sponsor associated with the patient and/or a patient identifier that identifies the patient to the plan sponsor. The member data may also include, by way of example, dispensation preferences such as type of label, type of cap, message preferences, language preferences, or the like.

The database 112D can represent a pharmacy database that stores pharmacy claims data. This claims data may be similar or identical to the claims data stored in the database 112A except related to claims submitted to pharmacies.

The database 112E can represent a plan database storing benefit plan eligibility data. The eligibility data can include claim adjudication rules or criteria used to decide whether a medication or treatment claim is covered by a benefit plan, whether certain providers are within a benefit network provided by the benefit plan, what details are needed for preauthorization of a benefit claim to be approved, what details are needed to appeal a denial of a claim, etc.

The database 112F can represent a documentation database that stores documents or logs. These documents can detail the eligibility data stored in the database 112E, can store logs from prior interactions between the agent device 102 and member devices 104 (e.g., a history of calls or messages between the devices 102, 104), prior actions taken in response to an inquiry from a member to the agent device 104, or the like.

The ANN 106 can access one or more of the databases 112 based on the recorded inputs and the context of the inquiry as determined by the ANN 106. The ANN 106 can locate and obtain one or more sets of responsive information from the healthcare-related databases 112 based on the inputs. For example, in response to the ANN 106 deciding that the inquiry is asking whether a prescription for medication or a treatment is covered by benefits under a benefit plan, the ANN 106 can access the database 112E to obtain eligibility data for the prescription and/or the database(s) 112A, 112B, 112D to see whether prior benefits were provided to the member or other member(s) pursuant to the plan.

As another example, the ANN 106 can determine that the prescription request is associated with one or more prerequisites. The prerequisites may include having tried one or more other different medications prior to the requested medication. For example, the prerequisite(s) may require the member to have first administered a first type of prescription medication prior to fulfilling the requested prescription medication. The ANN 106 can access the database(s) 112A, 112B, 112C, 112D to examine whether the member has satisfied all or some of any prerequisites for the requested prescription medication.

For example, the ANN 106 can examine whether the patient previously had prescribed, filled and/or administered the first type of prescription medication, the date at which the first type of prescription medication was filled and/or administered, the dosage, the prescribing physician, the pharmacy at which the first type of prescription medication was fulfilled, patient biometric data prior to and/or subsequent to administering the first type of prescription medication, medical notes and/or physician comments associated with a medical visit subsequent to the first type of prescription medication being filled and/or administered, or the like, in order to determine whether or not the patient has satisfied the prerequisites for approving the prescription request.

In response to the ANN 106 deciding that the inquiry seeks guidance on whether preauthorization for a benefit under the plan is required, the ANN 106 can access the database(s) 112A, 112B, 112D to examine prior similar or identical claims (e.g., for the caller, for different patients having similar and/or related personal health information, etc.) and decide whether preauthorization was required in those prior claims (as well as what information was needed to obtain the preauthorization). The ANN 106 can access the plan eligibility data to obtain information on whether preauthorization is needed and what information is required for obtaining the preauthorization from the database 112E, as well as the database 112F to examine prior histories of the member to see whether preauthorization was previously required for the benefit claim.

Once the information is gathered by the ANN 106, the ANN 106 can aggregate the data obtained from the databases 112, generate one or more electronic signals, and send the signal(s) to the agent device 102. These signals are sent to communicate or convey the set(s) of responsive information obtained by the ANN 106 to the agent device 102. This can provide a single source or interface for the agent device 102 to obtain the information that responds to the caller inquiry from the member device 104. In contrast, the agent using the agent device 102 may be required to successively access, in a serial manner, each database 112 containing (or that the agent believes may contain) information responsive to the inquiry. Accessing each database 112 can require separate login and password entries, clicking through multiple interfaces, and the like, to obtain the responsive information from that database 112 alone. Additionally, the agent may mistakenly interpret some of the medical notes and/or medical history in the databases, may mistakenly believe that a database 112 may have responsive information and spend additional time accessing and searching through that database 112 for the information that is not in the database 112, or the like. All of this can consume a significant amount of time.

In one or more examples, the ANN 106 can send a signal to the agent device 102 that directs the agent device 102 (e.g., an electronic display device of the agent device 102, such as a computer monitor, touchscreen, or the like) to present the summary to the call center agent via the agent device 102. The summary may include citations and/or locations in the databases 112 where at least some of the medical data was located. The summary may also include a recommendation as to whether the prescription request should be approved, the prescription request should be denied, or if any additional information is necessary in making the decision to approve or deny the request. Optionally, the summary may include one or more reasons or explanations as to the reasoning behind the recommendation. For example, the summary may indicate to the call center agent that the request should be denied because one or more patient medical biometric data points indicated that the patient had not satisfied one or more requirements associated with the prescription medication. As another example, the summary may indicate that at least one medical note in the medical history indicated that a physician had recommended that the patient first use a different medication other than the one being requested. As another example, the summary may include a recommendation for the call center agent to send communication to a physician to confirm a medical note discovered in the medical history, with citation to the medical note discovered in the databases.

The call center agent can then review the summary and either approve or reject the summary and/or the recommendation made by the ANN 106. For example, if the summary accurately reflects the interaction with the member, then the agent can provide input into the agent device 102 indicating validation or approval of the summary (referred to as a validation action). This validation action can be communicated to the ANN 106, which can then populate a call log with the summary and store the call log in the database 112F for further use in future calls. As another example, if a portion of the summary inaccurately reflects the interaction with the member, or if the agent disagrees with the recommendation made by the ANN 106, the agent may communicate the rejection or disagreement, and a reasoning for disagreeing with the summary or recommendation, with the ANN 106 via the agent device 102, which can then learn from the discrepancy between the ANN 106 generated summary and the call agents decision.

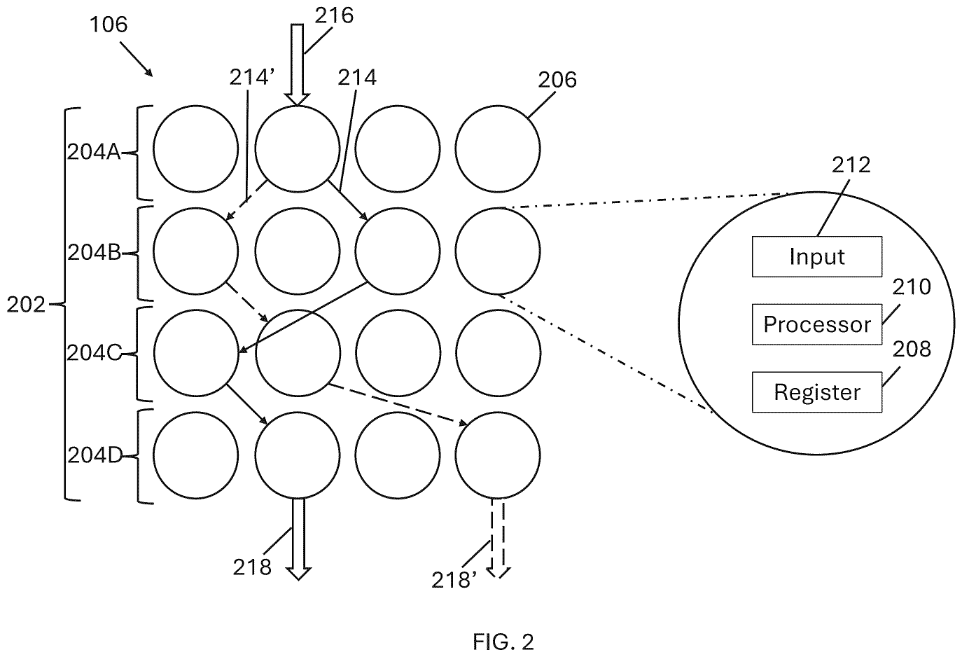

FIG. 2 illustrates one example of the ANN 106 shown in FIG. 1. The ANN 106 can be embodied in one or more ASICs for the ANN 106. The ASIC(s) can include a series 202 of layers 204A-D, each comprising one or more artificial neurons 206 arranged in one or more neuron arrays or arrangements. While four neurons 206 are shown in each layer 204A-D and four layers 204A-D are shown, alternatively, a different number of neurons 206 may be in one or more of the layers 204A-D and/or there may be a different number of layers 204A-D.

The ANN 106 may include the neurons 206 arranged in an input layer 204A, an output layer 204D, and two or more fully connected hidden or intermediate layers 204B, 204C between the input and output layers 204A, 204D. Each neuron 206 can include or represent a register 208, a microprocessor or processing element 210, and at least one input 212. The neurons 206 can generate output based on one or more activation functions. The neurons 206 can receive input from another neuron 206 (e.g., the output from one neuron 206 can be the input for another neuron 206). This input also can include a set of weights. The neurons 206 can be connected with each other via synaptic circuits 214, 214'. The synaptic circuits 214, 214′ can include or represent memories for storing synaptic weights.

The ANN 106 implements a call center operations system 100 that automates review of prescription requests by capturing interactions between the member device 104 and the agent device 102, processes these interactions through the layers 204A-204D, tokenizes the interactions, maps token sequences into a trained vector space to determine inquiry context, and selects which databases 112A-112F to query. The ANN 106 can receive a rejection of a prescription fill or refill as an input, processes the information collected from the databases 112, and outputs a recommendation to approve or deny the fill or refill. The use of the specially trained ANN 106 to perform these operations creates a number of improvements over traditional methods of approving or denying prescription fills or refills, including more accurate approval or denial decisions, as well as fulfilling the prescription fills or refills that are approved. The ANN 106 is trained as described herein, which leads to faster training times and a more accurate model for denying or approving (and filling) the prescriptions. The ANN 106 is a type of machine learning model used to perform a wide variety of complex tasks as described herein. The ANN 106 can be implemented through software, hardware, or a combination of software and hardware.

One or more neurons 206 in the input layer 204A of the ANN 106 can receive an input 216 into the ANN 106. The input 216 can include, for example, the recorded inputs of a call or interaction between the devices 102, 104 shown in FIG. 1. The neurons 206 can receive this input data 216 via the input(s) 212 of the neurons 206 in the input layer 204A. The neurons 206 receive the input data 216, apply one or more mathematical equations or relationships stored in the registers 208 (and that include the weights) to generate an output. The processors 210 of the neurons 206 apply the equations/relationships and can pass the output to another neuron 206 in the same layer 204A or in a different layer 204B, 204C. The output from one neuron 206 is passed along a synaptic circuit 214 to another neuron 206 and is used as input to this other neuron 206. This process continues until one or more neurons 206 in the output layer 204D generate an output 218 from the ANN 106.

The synaptic circuits 214, 214′, weights stored in the synaptic circuits 214, 214′, and/or the mathematical relationships between the neurons 206 can define the LLM 110. In operation, the ANN 106 can operate by converting the recorded inputs into a machine-readable summary. This conversion can be performed by modifying the recorded inputs into series of tokens via a tokenization process. The different tokens can represent words in the recorded inputs, portions of words such as morphemes, phrases formed of multiple words, and the like. The synaptic circuits 214, mathematical equations, and weights defining the model 110 may dictate what tokens are generated based on the recorded inputs. Changing the model 110 by changing which neurons 206 are connected by which synaptic circuits 214, which weights are applied to different words or inputs into the recorded inputs to select which words or phrases are included in the tokens, and the like, can change the sequence of tokens output based on the recorded inputs. For example, the LLM 110 can output a first sequence of tokens but output a different, second sequence responsive to the LLM 110 being modified (e.g., refined).

The ANN 106 can then map the token sequence into vector space. During training of the LLM 110, the ANN 106 using the LLM 110 can encounter words and examine the different contexts of the words as well as relationships with other words. ANN 106 can assign a unique vector to each word in the training data based on the LLM 110 during this training. These single word assignments can be used by the ANN 106 to create assignments of text documents to unique vectors in the vector space. After training, the ANN 106 can use the LLM 110 to assign different vectors to different sequences of tokens. Each vector can define a location within the vector space, and words and/or phrases with similar or identical meanings may have locations that are closer in the vector space than words and/or phrases with more dissimilar or different meanings. Additionally, the distance and direction between vectors within the vector space can represent relationships between the words and/or phrases indicated by the token sequences. For example, the distance and direction between vectors may reflect semantic relationships between the words and/or phrases associated with the token sequences.

This vector space may then be used by the ANN 106 to identify context of inquiries from member devices 104, as well as to determine what responsive information is needed from the databases 112. For example, certain vectors are associated with different sets of responsive information. If the tokens from a recorded input is closer to one vector than others in the vector space defined by the LLM 110, then the ANN 106 can obtain the responsive information from the databases 112 that are associated with that vector. The LLM 110 can be refined based on feedback. For example, if the returned responsive information is not responsive to the inquiry, feedback identifying the additional or different responsive information that is needed can be provided to the ANN 106. The ANN 106 can then modify the circuits, weights, or the like, of the LLM 110 so that a different vector is associated with the same recorded input as before. This can allow the LLM 110 to continue to improve over time.

As time continues, the call center agent will have continued interactions with the ANN 106 regarding prescription requests. The ANN 106 can use the deep history of data maintained over time from interactions between patients and the call center operations system 100, providers, pharmacies, etc., to assist the call center agent with future prescription medication requests.

In one or more embodiments, the LLM 110 may continuously or repeatedly (e.g., at random or scheduled intervals of time) monitor one or more of the databases 112 for one or more updates or changes made to any existing medical data records, or for the addition of any new medical data records associated with the caller. As one example, one or more data records within one of the databases may be changed, updated, or added to the database while the caller is communicating with the call center agent. As another example, the data records may be changed, updated, or added to the database after communication between the caller and the call center agent has ended. In one or more embodiments, the ANN 106 may automatically communicate a notification to the call center agent indicating that the record(s) have changed or that new records are available. The communication may include an updated summary of the medical history of the caller, an updated recommendation based on the updated medical records available, or the like.

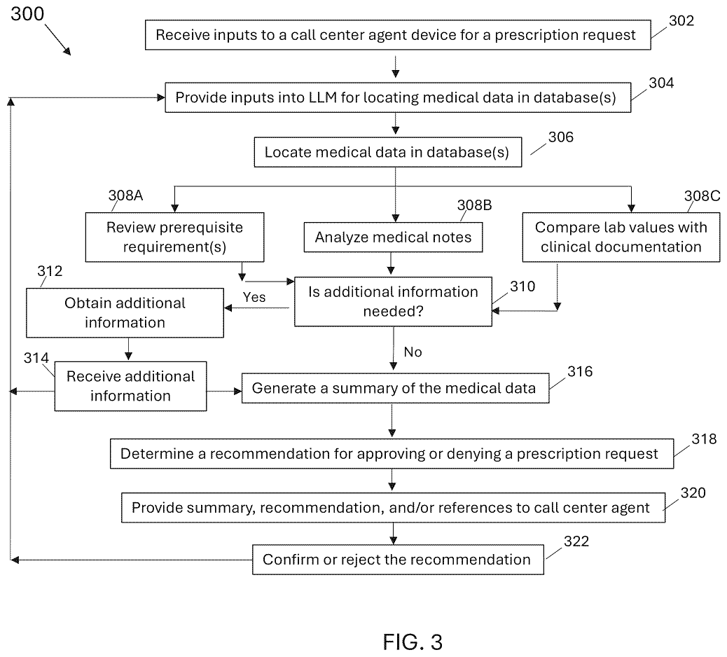

FIG. 3 illustrates a flowchart 300 of one example of a method for reviewing a prescription request at a call center. The method can represent operations performed by the operations system 100 shown in FIG. 1. At 302, inputs at a call center agent device are recorded. As described above, these inputs can include a transcription of the call, a log of keystrokes made by the call center agent, tools or applications accessed by the agent using the agent device, and so on. At 304, the inputs are provided into an LLM. The inputs can be provided into the LLM for the LLM to identify the context of the inquiry from the member contacting the agent, as well as to identify what information is needed to respond to the inquiry.

At 306, the database(s) containing the information that may be relevant to responding to the inquiry are accessed (e.g., by the ANN) and medical data associated with the inquiry may be located and analyzed by the one or more processors of the ANN. Following 306, one or more operations 308A, 308B, 308C can be performed. For example, at 308A, one or more prerequisites associated with the medication being requested may be reviewed. The prerequisites may include, but are not limited to, one or more medical test results of the caller, one or more medications being previously prescribed and/or administered, follow-up office visits with a physician, or the like. At 308B, the ANN can analyze medical notes located with the databases. The medical notes may be manually entered and/or freely written by a medical professional. In one or more embodiments, the ANN may include and/or have access to an optical character recognition (OCR) model that can read the medical notes and provide a summary of the medical notes to the call center agent. At 308C, lab values and/or medical test results may be compared with clinical documentation, medication requirements, or the like. For example, biometric lab values located in the databases may be compared with standard and/or baseline values associated legislative/regulatory requirements (e.g., minimum and/or maximum values directed by the Food and Drug Administration FDA, values directed by a benefit plan, or the like).

At 310, a determination is made by the ANN if any additional information is needed in generating the summary and/or providing the recommendation to the call center agent. If no additional information is needed, flow of the method proceeds toward 316. Alternatively, if any additional information is needed, flow of the method proceeds toward 312.

At 312, additional information is obtained. As one example, the ANN may communicate with the call center agent via the agent device what additional information may be needed in making the recommendation to approve or deny the prescription request. The communication to the call center agent may provide instructions for the agent to ask the contacting member for the additional information, may instruct the agent to contact a physician, a pharmacist, or other medical professional to confirm and/or clarify some of the medical data located in the databases or to provide information that was missing from the databases, or the like. As another example, the ANN may contact the physician, the pharmacist, or other medical professional directly requesting the additional information.

At 314, the additional information is received by the ANN, such as being provided by the call center agent via the agent device or being provided by the medical professional contacted by the ANN.

At 316, a summary of the historical medical data is generated. The summary may be based on the medical history of the contacting member including patient data, historical prescription and/or non-prescription medication data, medical notes by the medical professional, or the like. In one or more embodiments, the summary may include reference to where in the databases the medical data was located and/or may identify the source of the medical data (e.g., test results, office visit, medical note, etc.).

At 318, a recommendation is determined for approving or denying the prescription request. The ANN may automatically determine the recommendation based on the historical medical data located in the databases, based on historical recommendations for the calling member or other calling members inquiring about the same or a similar prescription medication, or the like. As one example, the recommendation by the ANN may be to deny the prescription request based on a determination that at least one prerequisite was not satisfied. Alternatively, the recommendation by the ANN may be to approve the prescription request based on a determination that the calling member has satisfied the prerequisites associated with the prescription medication being requested.

At 320, signals are generated and communicated to the call center agent device with the summary, the recommendation, and/or citation references. For example, the ANN can send the information responsive to the inquiry to the agent device.

At 322, the call center agent can confirm or reject the recommendation generated by the ANN. As one example, the ANN recommendation may be to deny the prescription request, but the call center agent may override the denial based at least on some of the information provided in the summary of the medical history of the calling member and approve the prescription medication request. Alternatively, the ANN recommendation may be to approve the request, but the call center agent may override the approval and deny the prescription medication request. In one or more embodiments, the call center agent can then present or provide the information to the patient (e.g., via the member device), to a physician of the patient, or the like, advising the approval or denial of the prescription medication request.

In one or more embodiments, flow of the method may return to 304 in which the LLM can then learn from the discrepancy between the ANN generated summary and recommendation and the call agents decision.

While the operations described in connection with FIG. 3 are shown in a sequential order, one or more of these operations may be skipped and/or the order in which the operations are performed may be swapped. In another example, two or more of the operations may be performed concurrently or simultaneously, one or more operations may be skipped or omitted, one or more operations may be duplicated, or any combination therein.

The inventive subject matter described herein relates to specific, practical applications of technology implemented with particular computer technology that improves the functioning of the computer systems themselves. The preceding disclosure describes the LLM system 110 operating in conjunction with an ANN 106 embodied in defined layers 204A-204D with synaptic circuits 214, 214′, neurons 206 having registers 208, processing elements 210, and inputs 212, implemented in one example in one or more ASICs. The system 100 processes heterogeneous medical data from multiple networked databases 112A-112F via computerized communication networks and recorded inputs 216 from the call center agent device 102 interacting with the member device 104. The ANN 106 performs tokenization of recorded inputs, maps sequences into a trained vector space to identify the context of inquiries, accesses the particular databases 112A-112F to locate medical data including patient data, historical prescription data, and medical notes, applies optical character recognition to free-text clinical notes, and generates machine-readable outputs 218 comprising a summary and a recommendation that are automatically communicated to the agent device 102 and, in certain examples, to automated and manual pharmacy fulfillment devices. This particularized combination of components and operations is necessarily rooted in computer technology and materially improves the speed, accuracy, and efficiency of prescription-request adjudication.

Additionally, the subject matter described herein is directed to a specific technical solution to a technological problem. Specifically, the problem of efficiently ingesting, normalizing, and interpreting large volumes of disparate medical data across multiple sources to automate call-center workflows. The recited architecture of the ANN 106, including the layers 204A-204D, neurons 206, and synaptic circuits 214, 214′, the tokenization and vector-space mapping performed by LLM system 110, and the OCR processing of unstructured clinical notes in databases 112B and 112F are concrete data-processing techniques that cannot be performed in the human mind and that are implemented by particular machine components as described herein. The operations transform unstructured, multi-format medical inputs (e.g., claims in databases 112A and 112D, provider data and clinical notes in the database 112B, member data in the database 112C, eligibility rules in the database 112E, and documentation logs in the database 112F) into a structured summary and recommendation through specific model-driven processing steps. The outputs 218 are then used to generate signals to display devices associated with the agent device 102 and can drive downstream actions such as pharmacy fulfillment processes, demonstrating improvement in computer functionality for medical data analysis.

The subject matter described herein also is integrated into one or more practical applications. The system 100 accesses multiple identified databases 112A-112F via computerized communication networks, processes the data using the particular LLM system 110 and the ANN 106 configuration with the neurons 206, registers 208, processors 210, and synaptic circuits 214, 214′, generates signals that cause display of the summary and recommendation on the agent device 102, and, in some examples, drives automated dispensing equipment and fulfillment systems to execute approved prescriptions, including packaging, labeling, and shipping as described for pharmacy fulfillment devices. The LLM system 110 additionally performs continuous or repeated monitoring by the LLM system 110 for updates to medical records in the databases 112, as well as automatic notifications with updated summaries and recommendations provided to the agent device 102. These features tie the data-processing steps to real-world medical and pharmacy operations, impose meaningful limits, and use technology to effect a physical distribution of prescription medications, which constitutes one or more practical applications.

The subject matter described herein also provide inventive concepts that are significantly more than any abstract idea. The specific combination of the LLM system 110 with the ANN 106 implemented with the synaptic circuits 214, 214′, weights stored therein, and the neuron layers 204A-204D; tokenization and mapping into a trained vector space for context-aware retrieval across the healthcare databases 112A-112F; OCR of physician notes from the provider database 112B and the documentation database 112F; and automated communication of signals to fulfillment devices is not conventional or routine. The architecture as described herein, including inter-layer connections and flow of inputs 216 through the hidden layers 204B-204C to the outputs 218, is configured to perform the recited tasks in a manner that yields substantially improved performance relative to prior manual call-center workflows that required serial, human access to disparate systems. The subject matter described herein reduces errors arising from misinterpretation of medical notes, avoids wasted time accessing non-responsive databases, and accelerates determinations from hours to minutes by aggregating responsive information in the system 100 and presenting the information via the agent device 102. These improvements in speed, accuracy, and resource utilization reflect more than applying a generic computer to a business process. Instead, these improvements show a particularized technical implementation that provides benefits specific to the computer systems and networks involved.

Humans cannot practically or accurately perform the recited tokenization, vector-space mapping, and weight-based neural computations across massive, heterogeneous datasets in real time as executed by the neurons 206 and synaptic circuits 214, 214′. Additionally, humans cannot, without specialized machine components, perform OCR of free-form clinical notes stored in the databases 112B and 112F, integrate results with plan eligibility data in the database 112E, and propagate signals to pharmacy fulfillment devices. The operations of the system 100 as described herein require one or more of processors 210, memories including registers 208 and synaptic memories, the LLM system 110, the ANN 106, and/or the networked databases 112A-112F. Additionally, the system 100 produces the machine-generated outputs 218 that drive subsequent computerized or physical actions. As such, the inventive subject matter is not limited to merely collecting and analyzing information. Instead, the inventive subject matter effectuates control signals within the system 100 and integrates with fulfillment workflows, demonstrating application beyond mere data manipulation.

The subject matter described herein also provides a feedback refinement loop in which the ANN 106 modifies circuits, weights, or vector associations based on discrepancies between an ANN-generated summary and the call center agent's decision received via the agent device 102. As described, the flow of the method 300 can return from 322 to 304, which involves changing and improving the model using the recorded inputs 216 and outputs 218. This technical feedback mechanism enhances the accuracy of the model and reduces future misclassification, which improves the capability of the computerized system 100 to identify context and retrieve responsive information across diverse medical sources. These improvements to token selection, vector associations, and synaptic weights change how the system 100 operates at a fundamental level and are not ancillary to a business objective.

The subject matter described herein provides particular components and roles within the ANN 106, provides synaptic circuits 214, 214′ storing weights, details tokenization and vector-space assignments during training and inference by the LLM system 110, and describes OCR applied to physician notes in the databases 112B, 112F. At least one example of the inventive subject matter described herein requires a specific architecture configured to ingest, transform, and act upon healthcare data for prescription workflows, including interactions with the benefit claims database 112A, the provider database 112B, the member database 112C, the pharmacy database 112D, the plan eligibility database 112E, and the documentation database 112F. Generation of signals to the agent device 102 and optional pharmacy fulfillment devices demonstrates an unconventional arrangement of technological elements that addresses the unique challenges of healthcare data heterogeneity and call-center decision support.

One or more examples of the system 100 is tied to a particular machine, specifically the LLM system 110, the ANN 106 with layers 204A-204D, neurons 206, and synaptic circuits 214, 214′, the memory 108 storing models and recorded inputs, the agent device 102 and member device 104, and the networked databases 112A-112F. The method 300 includes operations that affect a transformation of unstructured inputs 216 into actionable, machine-readable outputs 218 that alter the state of a prescription-fulfillment system.

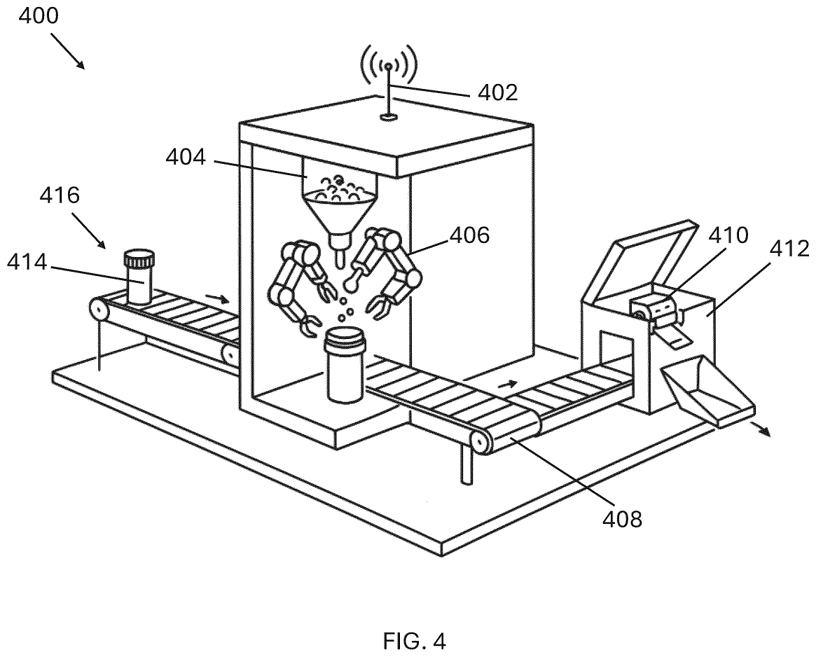

FIG. 4 illustrates one example of a pharmacy fulfillment device 400 that can be used to process and fulfill prescriptions and prescription orders. The pharmacy fulfillment device 400 can receive the signal from the ANN 106 indicating approval of the prescription fill or refill, or this signal may be received indirectly from the ANN 106 (via the agent device 102). The prescriptions may be automatically filled by the pharmacy fulfillment device 400 according to dispensation preferences of the calling member, such as type of label, type of cap, message preferences, language preferences, or the like, identified in the database 112C.

The pharmacy fulfillment device 400 may include a communication device 402 in communication with the ANN 106 and/or the agent device 102. For example, the communication device 402 can exchange signals with the ANN 106 and/or the agent device 102 to coordinate automated prescription fulfillment operations based directly on the output of the ANN 106. The communication device 402 can receive, directly from the ANN 106 or indirectly from the ANN 106 via the agent device 102, control signals generated in response to a validated recommendation and summary output 218 from the ANN 106. The communication device 402 can receive (from the agent device 102) operator approvals, overrides, and fulfillment preferences associated with a particular prescription request. The communication device 402 can authenticate the source of the signals, parse prescription identifiers, dispensation parameter, packaging requirement, and labeling content, and route corresponding instructions to subsystems of the pharmacy fulfillment device 400, such as an automated dispensing device 404, a robotic pick-and-place system 406, a conveyor 408, a printing module 410, and/or a shipping device 412. The communication device 402 can further transmit status updates and completion confirmations back to the ANN 106 and/or the agent device 102 to provide closed-loop monitoring of fulfillment progress, exception handling, and synchronization of documentation in the databases 112A-112F. In some examples, the communication device 402 periodically polls for updates from the ANN 106, such as modified recommendations or revised patient preferences stored in the memory 108, and can dynamically adjust ongoing automated fulfillment tasks within the pharmacy fulfillment device 400 to ensure compliance with plan eligibility data in the database 112E and dispensation preferences in the database 112C.

For example, the ANN 106 and/or the call center agent 102 may communicate the confirmation to approve the prescription medication request to the pharmacy fulfillment device 400 to begin the fulfillment process of the medication. This communication may include one or more instructions for packaging the prescription medication according to one or more fulfillment requirements and/or preferences of the patient; for printing labels for the packaging; or for shipping the packaged prescription according to one or more shipping requirements, shipping preferences of the patient, or shipping requirements outlined by the benefits plan of the patient.

The pharmacy fulfillment device 400 can automatically operate based on the machine-generated output 218 provided by the ANN 106 and, in some examples, confirmations issued via the agent device 102. Upon receipt of fulfillment instructions, the conveyor 408 advances a medication container 414 from a staging area 416 to a position directly beneath the automated dispensing mechanism 404 (as shown in FIG. 3). The automated dispensing mechanism 404 can then meter a prescribed quantity of medication identified in the prescription request into the container 414 according to parameters included in the output 218 provided by the ANN 106, such as drug identifier, dose, and count. During dispensing, optical sensors or other sensors associated with the automated dispensing mechanism 404 may optionally corroborate that the correct medication and quantity are delivered and can provide feedback to the pharmacy fulfillment device 400 to halt or adjust the operation if a discrepancy is detected.

After dispensing, the robotic pick-and-place system 406 can maneuver the container 414 to apply a closure to the container 414, such as a cap specified by patient preferences stored in databases 112C, and can engage a sealing routine to provide tamper resistance. The robotic pick-and-place system 406 can further orient the closed container 414 for downstream processing, such as by placing the container 414 in a stable posture on the conveyor 408 and signaling completion of the closure step back to the pharmacy fulfillment device 400.

Following closure, the conveyor 408 transports the filled and sealed container 414 to the printing module 410. The printing module 410 can represent hardware configured to receive prescription-specific information from the ANN 106 and print directly onto the container 414 or onto a label that is adhered to the container 414. The module 410 can include an onboard controller with one or more processors and memory, a thermal or thermal-transfer print engine that produces text and barcodes, and an applicator mechanism (e.g., a wipe-on, tamp-blow, or wrap-around applicator).

The printing module 410 prints and applies a label to the container 414 (or prints directly onto the container 414) using information included in the output 218 from the ANN 106 and associated records retrieved from the databases 112A-112F, such as patient identifiers, medication name, dosage instructions, refill counts, and plan eligibility notices. The printing module 410 can verify label adhesion and readability and can reprint or reapply a label when needed. The printing module 410 can then eject or otherwise output the filled, sealed, and labeled container 414 into the shipping device 412.

The shipping device 412 receives the filled, sealed, and labeled container 414 and packs the container 414 into a box or other shipping vessel for delivery to the patient. The shipping device 412 can represent a programmable motion controller, one or more pick-and-place actuators, an infeed conveyor section mechanically coupled to the conveyor 408, optionally a carton erector, and/or a scale. The motion controller can coordinate receipt of the labeled container 414 from the label printing module 410. The carton erector can erect a shipping vessel, place cushioning material, and deposit the container 414 into the vessel. The shipping device 412 can include a barcode or RFID scanner for container 414 verification, a printer for printing shipping labels, and a sealing module (e.g., a tape head or hot-melt applicator) to close the vessel.

The shipping device 412 can select packaging materials and shipping methods based on fulfillment requirements contained in the output 218 from the ANN 106 and/or preferences stored in the database 112C, and can generate tracking data and shipment confirmations. Upon completion of packing, the shipping device 412 transmits a status update to the pharmacy fulfillment device 400, which can be relayed to the ANN 106 and/or the agent device 102 to confirm filling of the prescription. In this manner, the coordinated operation of the conveyor 408, automated dispensing mechanism 404, robotic pick-and-place system 406, printing module 410, and shipping device 412 provides an end-to-end, automated prescription fulfillment process driven by the outputs generated by the ANN 106. This coordinated operation is initiated and controlled by the output 218 generated by the ANN 106. The output 218 specifies, for example, medication identity, dosage, quantity, container type, closure requirements, labeling content, and shipping parameters, and these machine-readable instructions are parsed by the pharmacy fulfillment device 400 to sequence and synchronize operation of the conveyor 408, the dispensing device 404, the pick-and-place system 406, the printing module 410, and the shipping device 412. Without the output 218 from the ANN 106, the pharmacy fulfillment device 400 lacks the prescription-specific control directions required to position the container 414 beneath the automated dispensing mechanism 404 via the conveyor 408, to actuate the robotic pick-and-place system 406 for container 414 alignment and closure, to provide compliant labels at the printing module 410, and to select packaging and generate shipment details at the shipping device 412. As a result, operation of the pharmacy fulfillment device 400 is contingent upon, and cannot proceed in the absence of, the output 218 from the ANN 106, which is critical and required to configure, trigger, and complete the automated fulfillment sequence in at least one example of the inventive subject matter described herein.

As another example, the call center agent may communicate with a manual fulfillment device (not shown) that may control how prescriptions are manually fulfilled responsive to the prescription request being approved. For example, the manual fulfillment device may receive or obtain a container and enable fulfillment of the container by a pharmacist or pharmacy technician. In some implementations, the manual fulfillment device provides the filled container to another device in the pharmacy fulfillment devices to be joined with other containers in a prescription order for a user or member. In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting capsules, tablets, or pills may be at least partially automated (such as through use of a pill counter). Prescription drugs dispensed by the manual fulfillment device may be packaged individually or collectively for shipping, or may be shipped in combination with other prescription drugs dispensed by other devices in the high-volume fulfillment center.

The agent device may communicate the one or more signals to the automated and/or manual fulfillment device responsive to the agent confirming the approval of the prescription request. The signals may include packaging requirements and/or preferences of the patient. In one example, the labels that may be printed for the packaging may include an indication of the approval of the prescription request confirmed by the call center agent. Optionally, the recommendation by the ANN 106 may include a number of refills that may be available to the patient before future requests may need to be approved. The labels printed for the packaging of the prescription medication may include the number of available refills based on the recommendation by the ANN 106 and approval of the call center agent.

In one example, a method for automatically reviewing a prescription request using a large language model (LLM) system is provided. The method can include receiving, by the LLM system, a request to locate medical data associated with a medical history of a patient from one or more data sources. The medical history can include patient data, historical prescription data, and medical notes provided by a medical professional. The LLM system can provide the medical data that is located by the LLM system to one or more processors. A summary of the medical history of the patient can be generated by the one or more processors, and a recommendation for approving or denying the prescription request can be determined based at least in part on the summary of the medical history of the patient.

The method can further include displaying, with a display device, the summary of the medical history and the recommendation to an operator. The recommendation by the processor can be to deny the prescription request, and the operator can override the recommendation that is determined, approve the prescription request, and fulfill the prescription request. Optionally, fulfilling the prescription request may include one or more of: communicating approval of the prescription request to the patient and/or a physician of the patient, packaging the prescription according to one or more fulfillment requirements, printing labels for the packaging, and/or shipping the packaged prescription. In one or more embodiments, the summary may include one or more references identifying the one or more data sources from where the medical data was located.

The method can further include identifying that the prescription request is for a first prescription, determining that the first prescription has one or more prerequisites, determining that the patient has not satisfied at least one of the one or more prerequisites for the first prescription based in part on the historical prescription data of the patient, and determining the recommendation to deny the prescription request for the first prescription. Alternatively, the method can further include identifying that the prescription request is for a first prescription, determining that the first prescription has one or more prerequisites, determining that the patient has satisfied the one or more prerequisites for the first prescription based in part on the historical prescription data of the patient, and determining the recommendation to approve the prescription request for the first prescription.

The method can further include analyzing, by the one or more processors, the one or more medical notes of the medical history of the patient. The medical notes may be manually entered by the medical professional. A summary of the medical notes may be generated. Optionally, the summary of the medical notes may be provided to an operator of a call center operations system.

A system for reviewing a prescription request is provided. The system can include a large language model (LLM) system that can receive a request to locate medical data associated with a medical history of a patient from one or more data sources. The medical history can include patient data, historical prescription data, and medical notes provided by a medical professional. The system can further include one or more processors that can receive the medical data that is located by the LLM system. The processors can generate a summary of the medical history of the patient and determine a recommendation for approving or denying the prescription request based at least in part on the summary of the medical history of the patient.

The system may further include a display device that can display the summary of the medical history and the recommendation to an operator. Optionally, the processors can identify that the prescription request is for a first prescription having one or more prerequisites and determine that the patient has not satisfied at least one of the one or more prerequisites based in part on the historical prescription data of the patient. The processors can determine the recommendation to deny the prescription request for the first prescription. Alternatively, the processors can identify that the prescription request is for a first prescription having one or more prerequisites and determine that the patient has satisfied the one or more prerequisites based in part on the historical prescription data of the patient. The processors can determine the recommendation to approve the prescription request for the first prescription.

Optionally, the processors can analyze the one or more medical notes of the medical history of the patient and generate a summary of the medical notes. The medical notes may be manually entered by the medical professional. The processors can provide the summary of the medical notes to an operator.

A method for automatically reviewing a prescription request using a large language model (LLM) system is provided. The method can include receiving, by the LLM system, a request to locate medical data associated with a medical history of a patient from one or more data sources. The medical history can include patient data, historical prescription data, and medical notes provided by a medical professional. The method can further include providing, by the LLM system, the medical data that is located by the LLM system to one or more processors. The one or more processors can generate a summary of the medical history of the patient and determine a recommendation for denying the prescription request based at least in part on the summary of the medical history of the patient. A display device can display the summary of the medical history and the recommendation to an operator to deny the prescription request. The recommendation that is determined by the processors can be overridden and approved by the operator, and the prescription request can be fulfilled.

The method can further include the operator confirming the recommendation for denying the prescription request that is determined by the one or more processors. Optionally, the method can include communicating denial of the prescription request to the patient and/or a physician of the patient.

Optionally, fulfilling the prescription request can include one or more of: communicating approval of the prescription request to one or more of the patient or a physician of the patient; packaging the prescription according to one or more fulfillment requirements; printing labels for the packaging; or shipping the prescription.

The method can further include identifying that the prescription request is for a first prescription, determining that the first prescription has one or more prerequisites, determining that the patient has satisfied the one or more prerequisites for the first prescription based in part on the historical prescription data of the patient; and determining the recommendation to approve the prescription request for the first prescription. Optionally, the method can further include analyzing, by the one or more processors, the one or more medical notes of the medical history of the patient. The medical notes may be manually entered by a medical professional. A summary of the medical notes may be generated.

The present system and methods can be used with various automated pharmacy fulfillment systems, e.g., those described in U.S. Pat. No. 12,094,009, granted 17 Sep. 2024; U.S. Pat. No. 10,332,061, granted 25 Jun. 2019; U.S. Pat. No. 10,294,029, granted 21 May 2019; and U.S. Pat. No. 9,697,335, granted 4 Jul. 2017, which are all hereby incorporated by reference. With reference to the U.S. Pat. No. 12,094,009 patent, the present system can process its prescription request review in parallel with or prior to the processing in the '009 patent. With reference to the U.S. Pat. No. 10,294,029 patent, its control unit waits to receive a signal from or dependent on the signal from the present LLM system prior to processing a prescription order. Other uses of the signal from the present system can be used to trigger operation of the fulfillment devices in the incorporated drug fulfillment systems.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described examples (and/or aspects thereof) can be used in combination with each other. In addition, many modifications can be made to adapt a particular situation or material to the teachings of the various examples of the disclosure without departing from their scope. While the dimensions and types of materials described herein are intended to define the aspects of the various examples of the disclosure, the examples are by no means limiting and are exemplary examples. Many other examples will be apparent to those of skill in the art upon reviewing the above description. The scope of the various examples of the disclosure should, therefore, be determined with reference to the appended claims, along with the full scope of equivalents to which such claims are entitled. In the appended claims and the detailed description herein, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various examples of the disclosure, including the best mode, and also to enable any person skilled in the art to practice the various examples of the disclosure, including making and using any devices or systems and performing any incorporated methods. The patentable scope of the various examples of the disclosure is defined by the claims, and can include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. A method for automatically reviewing a prescription request using a large language model (LLM) system, the method comprising:
receiving, by the LLM system, a request to locate medical data associated with a medical history of a patient from one or more data sources, the medical history including patient data, historical prescription data, and medical notes provided by a medical professional;
providing, by the LLM system, the medical data that is located by the LLM system to one or more processors;
generating, with one or more processors, a summary of the medical history of the patient;
determining, with the one or more processors, a recommendation for approving the prescription request based at least in part on the summary of the medical history of the patient;
communicating a control signal to a pharmacy fulfillment device that indicates approval of the prescription request; and
responsive to receiving the control signal at the pharmacy fulfillment device:
moving a container using a conveyor to a position for automated dispensing of medication identified in the prescription request from an automated dispensing device into the container;
automatically dispensing the medication into the container from the automated dispensing device;
closing the container using a robotic pick-and-place system;
moving the container containing the medication and that is closed to a printing module;
printing onto the container or printing a label adhered to the container using a printing module;
moving the container from the printing module to a shipping device using the conveyor; and
packaging the container for shipping using the shipping device.

2. The method of claim 1, further comprising displaying, with a display device, the summary of the medical history and the recommendation to an operator.

3. The method of claim 1, wherein the recommendation by the one or more processors is denying the prescription request, and further comprising:
overriding the recommendation that is determined by the one or more processors;
approving the prescription request by an operator; and
fulfilling the prescription request.

4. The method of claim 3, wherein fulfilling the prescription request includes one or more of:
communicating approval of the prescription request to one or more of the patient or a physician of the patient;
packaging the prescription according to one or more fulfillment requirements;
printing labels for the packaging; or
shipping the packaged prescription.

5. The method of claim 1, wherein the summary includes one or more references identifying the one or more data sources from where the medical data was located.

6. The method of claim 1, further comprising:
identifying that the prescription request is for a first prescription;
determining that the first prescription has one or more prerequisites;
determining that the patient has not satisfied at least one of the one or more prerequisites for the first prescription based at least in part on the historical prescription data of the patient; and
determining the recommendation to deny the prescription request for the first prescription.

7. The method of claim 1, further comprising:
identifying that the prescription request is for a first prescription;
determining that the first prescription has one or more prerequisites;
determining that the patient has satisfied the one or more prerequisites for the first prescription based at least in part on the historical prescription data of the patient; and
determining the recommendation to approve the prescription request for the first prescription.

8. The method of claim 1, further comprising:
analyzing, by the one or more processors, the one or more medical notes of the medical history of the patient, wherein the one or more medical notes are manually entered by the medical professional; and
generating a summary of the one or more medical notes.

9. The method of claim 8, further comprising providing the summary of the one or more medical notes to an operator.

10. A system for reviewing a prescription request, comprising:
a large language model (LLM) system configured to receive a request to locate medical data associated with a medical history of a patient from one or more data sources, the medical history including patient data, historical prescription data, and medical notes provided by a medical professional; and
one or more processors configured to receive the medical data that is located by the LLM system, the one or more processors configured to generate a summary of the medical history of the patient and determine a recommendation for approving or denying the prescription request based at least in part on the summary of the medical history of the patient.

11. The system of claim 10, further comprising a display device configured to display the summary of the medical history and the recommendation to an operator.

12. The system of claim 10, wherein the one or more processors are configured to identify that the prescription request is for a first prescription having one or more prerequisites, the one or more processors configured to determine that the patient has not satisfied at least one of the of the one or more prerequisites for the first prescription based at least in part on the historical prescription data of the patient and determine the recommendation to deny the prescription request for the first prescription.

13. The system of claim 10, wherein the one or more processors are configured to identify that the prescription request is for a first prescription having one or more prerequisites, the one or more processors configured to determine that the patient has satisfied the one or more prerequisites for the first prescription based at least in part on the historical prescription data of the patient and determine the recommendation to approve the prescription request for the first prescription.

14. The system of claim 10, wherein the one or more processors are configured to analyze the one or more medical notes of the medical history of the patient and generate a summary of the one or more medical notes, wherein the one or more medical notes are manually entered by the medical professional,
wherein the one or more processors are configured to provide the summary of the one or more medical notes to an operator.

15. A method for automatically reviewing a prescription request using a large language model (LLM) system, the method comprising:
receiving, by the LLM system, a request to locate medical data associated with a medical history of a patient from one or more data sources, the medical history including patient data, historical prescription data, and medical notes provided by a medical professional;
providing, by the LLM system, the medical data that is located by the LLM system to one or more processors;
generating, with one or more processors, a summary of the medical history of the patient;
determining, with the one or more processors, a recommendation for denying the prescription request based at least in part on the summary of the medical history of the patient;
displaying, with a display device, the summary of the medical history and the recommendation to an operator to deny the prescription request;
overriding the recommendation that is determined by the one or more processors and approving the prescription request by the operator; and
fulfilling the prescription request.

16. The method of claim 15, further comprising the operator confirming the recommendation for denying the prescription request determined by the one or more processors.

17. The method of claim 16, further comprising communicating denial of the prescription request to one or more of the patient or a physician of the patient.

18. The method of claim 15, wherein fulfilling the prescription request includes one or more of:
communicating approval of the prescription request to one or more of the patient or a physician of the patient;
packaging the prescription according to one or more fulfillment requirements;
printing labels for the packaging; or
shipping the prescription.

19. The method of claim 15, further comprising:
identifying that the prescription request is for a first prescription;
determining that the first prescription has one or more prerequisites;
determining that the patient has satisfied the one or more prerequisites for the first prescription based at least in part on the historical prescription data of the patient; and
determining the recommendation to approve the prescription request for the first prescription.

20. The method of claim 15, further comprising:
analyzing, by the one or more processors, the one or more medical notes of the medical history of the patient, wherein the one or more medical notes are manually entered by the medical professional; and
generating a summary of the one or more medical notes.
